(19) United States
(12) Patent Application Publication
(10) Pub. No.: US 2019/0298896 A1
(43) Pub. Date: Oct. 3, 2019

(54) BODY DRAINAGE APPARATUS

(71) Applicant: Tintron AB, VAXHOLM (SE)

(72) Inventor: Mikael CHARLEZ, Mölndal (SE)

(73) Assignee: Tintron AB, VAXHOLM (SE)

(21) Appl. No.: 16/307,977

(22) PCT Filed: Jun. 7, 2017
(86) PCT No.: PCT/SE2017/050606

§ 371 Date: Dec. 7, 2018

(30) Foreign Application Priority Data
Jun. 7, 2016 (SE) 1650793-1

(51) Int. Cl. A61M 1/00

(52) U.S. Cl. CPC A61M 1/0031; A61M 1/1039; A61M 1/0066; A61M 1/0037

(57) ABSTRACT

A body drainage system for draining fluid from a body cavity of a patient, the body cavity being provided with an access port, wherein the system comprises
a peristaltic pump mechanism,
a housing for housing at least a portion of the peristaltic pump mechanism,
a flexible tube unit configured to be connected at an access port end to the patient access port, and at a collection unit end to a collection unit,
a cover for covering movable parts of the peristaltic pump mechanism,
a regulator unit configured to regulate a rotational speed of the peristaltic pump mechanism,
wherein the flexible tube unit is configured to form a loop suitable to be placed in one way only at peristaltic pump mechanism making it possible for the peristaltic mechanism to compress a pump portion of the flexible tube unit repeatedly such that pumping occurs in a desired direction.

### TECHNICAL FIELD

The present invention relates generally to a drainage apparatus and a method for drainage of excess body fluid from a body cavity of a patient. More specifically it relates to a pumping device and a method for its regulation and control.

### BACKGROUND

In contemporary medical care, the movement of fluid from a body cavity to another point for collection is a routine need and can be performed in a number of ways. When tubing or piping is used for carrying the fluid during the movement either gravity or a pump is utilized to create and/or sustain a suction pressure needed to move the fluid from one point to another.

### SUMMARY OF THE INVENTION

At times the movement of fluid from the body must be performed in a gentle, slow and steady manner. Such gentle, slow and steady manner can be designated “peristalsis”. Peristaltic pumping may be performed in a number of ways including, but not exclusively, by hand pump or with the use of a peristaltic pump.

In medical care, drainage procedures are typically performed by hand pumping, to achieve the peristaltic movement of excess fluid in a patient's body into drainage bags for disposal or into syringes for laboratory analysis or any other medical use. Hand pumping is time consuming and requires a person to be in attendance at all times. Further, the attendant must manually perform the hand pumping necessary to sustain the peristaltic movement. It is difficult to generate consistent suction forces using hand pumping.

Additional known methods for drainage procedures include plastic vacuum suction bottles and wall/portable suction. These methods typically produce a constant suction rather than a peristaltic suction. These methods also include plastic bottles that are pre-assembled with a vacuumed pre-set under pressure causing inadequate suction; are bulky and causing storage, operational and shipping difficulties; typically, are limited in size necessitating frequent changes during the procedure; require special medical waste handling procedures; and when shattered in use create the danger of contamination problem of body fluids. Wall suction, in addition to providing only constant suction, is not readily available in all clinical settings. Wall units tend to create greater suction forces than what is safe for a normal drainage procedure.

Generally, there is provided an apparatus and a method for collection of a bodily fluid, the apparatus comprises a peristaltic pump device and the method comprises steps for the regulation and control of the peristaltic pump movements and collection of the bodily fluid.

According to a first aspect here is provided a drainage apparatus comprising:
an incoming flow connector,
a flexible tube, connectable to a patients access port; and
a peristaltic pump,
   wherein the flexible tube is configured to cooperate with the peristaltic pump, and
holding means that can be set in an open position, and in a closed position, respectively, and when closed holds the flexible tube against the peristaltic mechanism, contributing to an optimal peristaltic condition, and
a regulator to regulate the peristaltic movement of said fluid.

The body fluids that can be drained with the apparatus described herein includes serum, sputum, water, wound liquid, lymphatic liquid, extravascular blood, ascites fluid, fluid including proteins or a combination thereof.

According to a second aspect there is provided a method of utilizing a regulated peristaltic pump, tubing and collection unit for the peristaltic transportation and collection of fluid the method comprising:
evacuation of a bodily fluid from a bodily cavity via an access port;
draining the fluid away from the bodily cavity through the access port,
regulation of the applied peristaltic suction pressure via a regulator and guided by a peristaltic movement indicator;
draining the fluid away from the access port with a flexible tube;
pumping the fluid in a peristaltic manner; and
receiving the fluid in a collection unit whereas said fluid properties, such as pH and lactate, are measured

According to a third aspect there is provided a kit of parts suitable for the drainage of a body fluid from a patient, the kit comprising:
a peristaltic pump unit, having a rotatable peristaltic mechanism, and first fixation means for a loop of drainage tubing;
a loop of drainage tubing configured to fit at least partially around the peristaltic mechanism, and being provided with a second fixation means for cooperating with the first fixation means of the peristaltic pump unit in order to keep the drainage tubing in position during pumping;
a collection unit for collection the drained fluid

### BRIEF DESCRIPTION OF THE DRAWINGS

In order that the manner in which the above recited and other advantages and objects of the invention are obtained will be readily understood, a more particular description of the invention briefly described above will be rendered by reference to specific embodiments thereof which are illustrated in the appended drawings.

Understanding that these drawings depict only typical embodiments of the invention and are not therefore to be considered to be limiting of its scope, the invention will be described and explained with additional specificity and detail through the use of the accompanying drawings in which:

FIG. 1a shows a front view of a drainage pump unit of a drainage system according to an embodiment of the present invention.

FIG. 1b shows an overview of drainage tube and collection unit of a drainage system according to an embodiment of the present invention.

FIG. 2a shows a schematic side view of a drainage apparatus with a cubic mechanism cover, together with a side and a top view of the mechanism cover.

FIG. 2b shows a schematic side view of a drainage apparatus with a cuboid mechanism cover, together with a side and a top view of that mechanism cover.

FIG. 2c shows a schematic side view of a drainage apparatus with a slidable mechanism cover, together with a side and a top view of that mechanism cover.

FIG. 2d shows a schematic side view of a drainage apparatus with a hinged mechanism cover, together with a side and a top view of that mechanism cover.

FIG. 3a shows a detail of the slidable mechanism cover of FIG. 2c together with the pump mechanism.

FIG. 3b shows a loop of drainage tubing and first fixation means.

FIG. 3c shows second fixation means being part of pump unit to receive first fixation means of FIG. 3b.

FIG. 4 shows the tube loop of the drainage tubing mounted around the peristaltic mechanism

FIG. 5 shows fixation means of a drainage device for a 90-degree loop of drainage tubing

### DETAILED DESCRIPTION

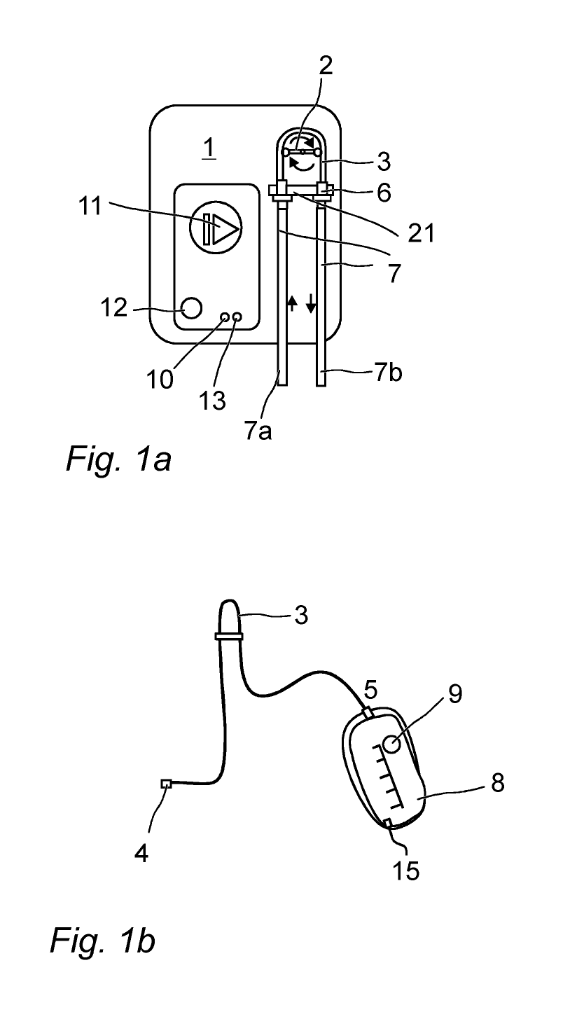

FIG. 1a and 1b shows a front view of a drainage apparatus according to an embodiment of the present invention. As seen in FIG. 1a, the drainage apparatus may comprise a housing 1 for housing major components of the apparatus, a connector 4 for connection the device to an access port of a patient, a flexible tube 3, and a peristaltic mechanism 2.

To use the drainage apparatus a patient's access port is connected to the incoming connector 4. The flexible tube 3 is inserted through the peristaltic mechanism 2 and is fixed in its operating position by first fixation means 6 that secure the operating position of said flexible tube 3 and secure optimal operating peristaltic conditions.

The apparatus is thus divided into a disposable portion comprising the flexible tube 3, the first fixation means 6 and the further drainage tube 7a and 7b. The drainage tube first portion 7a has a first end and a second end. The first end of the drainage first portion 7a comprises the incoming flow connector 4. The second end of the drainage first portion 7a is connected to the first fixation means 6.

In order to facilitate easy operation of the drainage apparatus the housing 1 is provided on its outer surface with a battery indicator 13 by which inspection to check if the device is fully loaded is readily performed.

The first fixation means 6 is placed in an open position to allow a person access, and the flexible tube 3 is inserted in its operating position guided by the fixation means 6 and then secured in the peristaltic mechanism 2 by the engaging the first fixation means 6.

A collection unit 8 is preferably provided and shall be connected to an out-coming connector 5. When the flexible tube 3 and the connectors, 4, 5 are in position the housing may be activated by pushing the power on button 12. Now the device is ready for connection to the patient's access port.

Pressing the peristaltic regulator button 11 activates the drainage procedure and the peristaltic pumping is indicated by the peristaltic movement indicator 10. As the bodily fluid is drained from the bodily cavity it enters the connector 4, the first portion 7a of the drainage tube 7 and the flexible tubing 3 and passes the peristaltic mechanism 2 and then to the out coming connector 5 and to the collection unit 8 via the second portion 7b of the drainage tube 7.

### Peristaltic Regulator

The peristaltic regulator 11 controls the peristaltic motor in a pre-programmed manner to perform what may be denoted a “drainage cycle” To provide this pre-programmed manner of control the peristaltic regulator 11 is configured to include an acceleration phase during which the peristaltic motor is controlled to accelerate from an rpm of zero revolutions per minute up to a predetermined operational rpm during a first predetermined time period.

The peristaltic regulator 11 is further configured to subsequently regulate the motor to keep the predetermined operational rpm during a second predetermined time period.

The peristaltic regulator 11 is further configured to subsequently, during a third predetermined time period, decelerate the peristaltic motor from the operational rpm down to an rpm of zero revolutions per minute.

The first predetermined time period may preferably be chosen in the interval of 20-40 seconds, the second predetermined time period may preferably be chosen in the interval of 150 to 250 seconds, and the third predetermined time period may preferably be chosen in the interval of 20-40 seconds. Most preferably the predetermined time periods are chosen as around 30, 200, and 30 seconds respectively.

The device is further provided with a peristaltic indicator 10. The peristaltic indicator 10 is configured to indicate to the user how much of the peristaltic movement capacity that has been utilized. The peristaltic mechanism is rotating and the regulator 11 controls the rotating rate. If the battery power supply reaches critical levels, the battery power indicator 13, alerts the user to recharge the battery.

Suction Pressure

The pressure regulator is preferably of simple, robust and cost-effective type. Preferably it is of non-feedback variety. Factory predetermined rpm limits secure effective but safe suction pressure.

As liquid is discharged through the out-coming connector 5 and transported through the second portion 7b of the drainage tube 7 and deployed in the collection unit 8, it will be in contact with the liquid property indicator 9, and liquid properties such as pH and Lactate are measured as well as the total accumulated volume.

Cylindrical Inner Surface

In various embodiments the system comprises a cylindrical or partially cylindrical inner surface, wherein the loop of the flexible tube unit 3, 6, 7 is configured to be placed in one way only at the rotatable peristaltic pump mechanism making it possible for the rotatable peristaltic mechanism to compress a pump portion (3) of the flexible tube unit (3, 6, 7) repeatedly against the cylindrical inner surface contributing to efficient pumping in the desired direction. The cylindrical inner surface is preferable an integral portion of the housing 1 or more preferred the cylindrical inner surface is an integral portion of the cover, such as a removable or slidable or hinged cover. However, the fixation means 6 are preferably sturdy enough to keep the flexible tube in place during action of the peristaltic mechanism even if there is no cylindrical inner surface to abut the cylindrical rolls of the peristaltic mechanism, or in the case such inner cylindrical surface has been broken or damaged.

Liquid Property Indicators

Further, the device may be provided with liquid property indicator(s) 9 to determine properties of the drained liquid. Liquid properties are determined with the aid of chemical indicator(s) provided at the inside of a transparent collection bag 8. A pH indicator could be of a halo chromic chemical compound so the acidity or basicity can be visually determined.

Furthermore, the said liquid property indicator 9 may determine the presence of lactate, reflecting metabolic stress via a test strip that contains the immobilized substrate, L-lactate, and be visually determined by its colour intensity.

The body drainage system may be configured to measure pH levels of the drainage fluid, by the system comprising electronic circuits to receive and process signals from pH sensors provided on an inner surface of the collection bag, and wherein the pH sensors are printed

The body drainage system may be configured to measure lactate levels of the drainage fluid, by the system comprising electronic circuits to receive and process signals from lactate sensors provided on an inner surface of the collection bag, and wherein the lactate sensors are printed.

The body drainage system is preferably provided with a display unit, and the system is configured to display pH and/or lactate measurements on the display unit. The display unit may be arranged on or as an integral part of the pump unit or pump casing, or may alternatively be arranged at a hanger for a collection bag for the drainage fluid.

Further, a sample port 15 is provided at a bottom end of the collection unit/collection bag to enable the user to take samples from the drained fluid for further testing.

The foregoing description of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of this invention as defined by the accompanying claims.

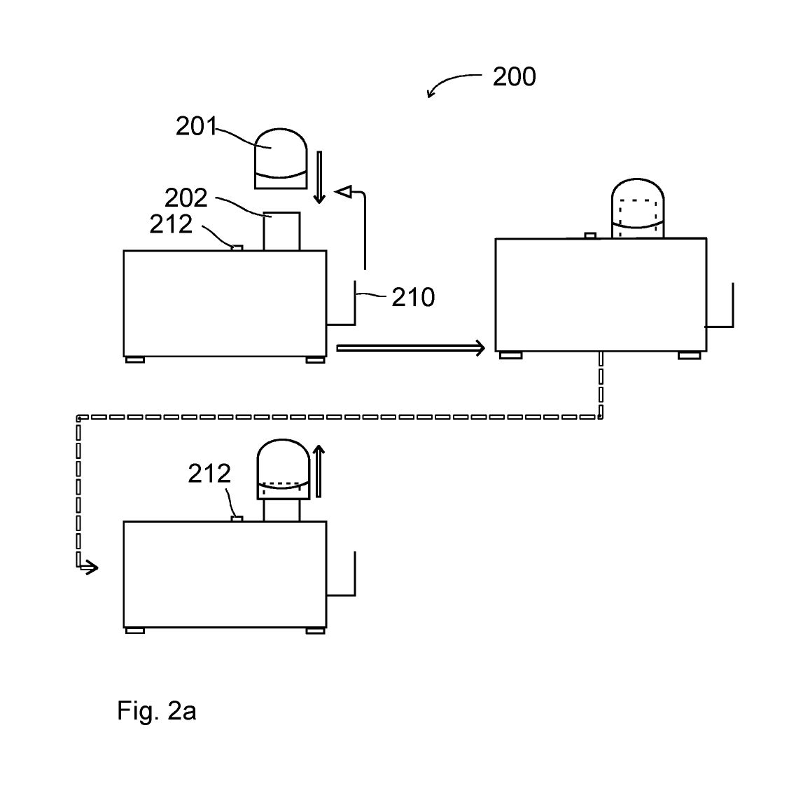

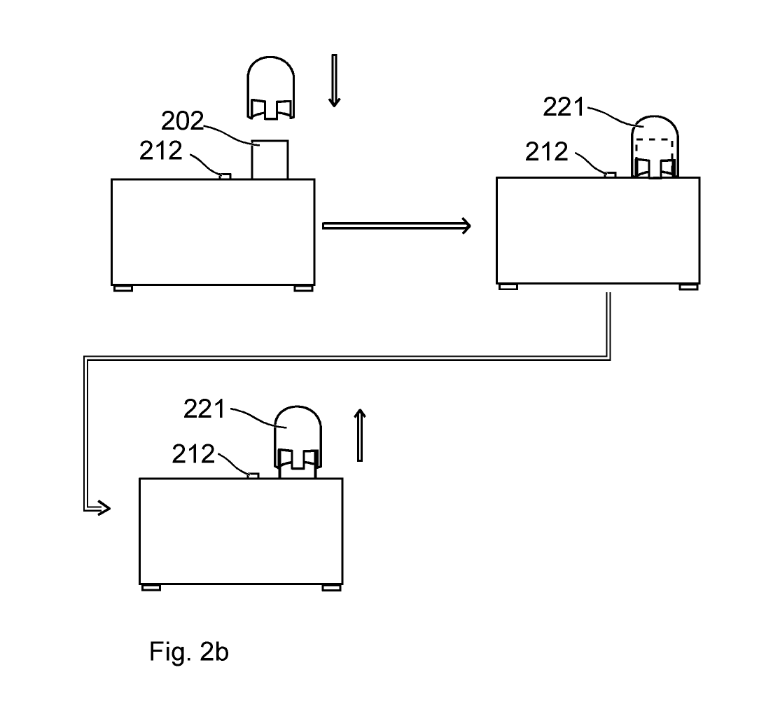

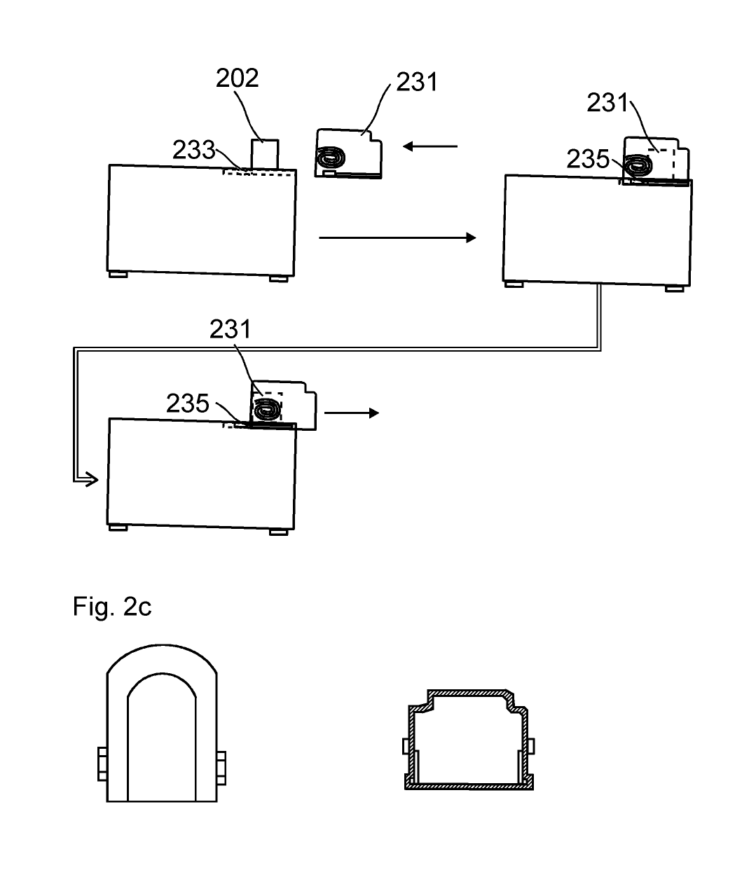

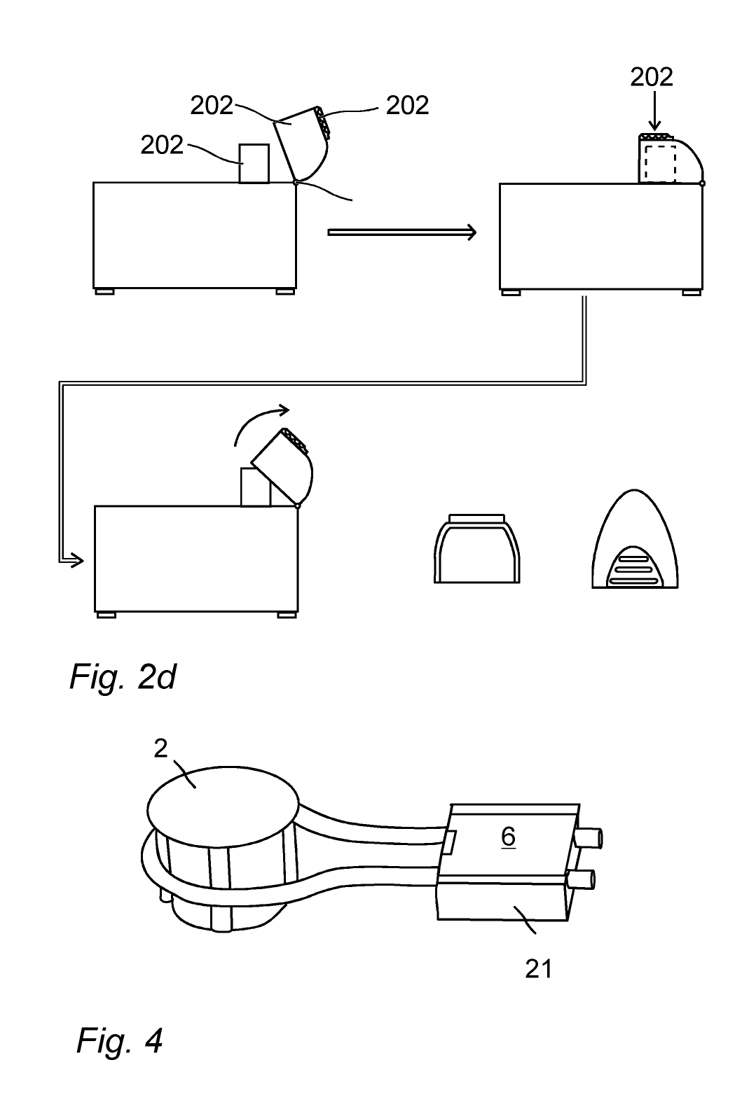

FIG. 2a shows a schematic side view of a drainage apparatus 200 with a cubic mechanism cover 201, together with a side and a top view of the mechanism cover 201. The cubic mechanism cover 201 is configured to be released by pushing a release button 212. When not in use the cubic mechanism cover 201 can be placed on a cover stand 210, preferably arranged at a short end of the unit 200.

The cubic mechanism cover 201 may preferably be provided with a switch that is configured to automatically turn off the pump when the cubic mechanism cover 201 is opened. The switch is preferably arranged with a mechanical lever at an edge of the cubic mechanism cover 201 and a contact arranged close to that edge of the cubic mechanism cover 201 such that when the cubic mechanism cover 201 is correctly placed over the mechanism the contact is closed, and when the cubic mechanism cover 201 is moved away from its correct position the contact is opened and a pump circuit is broken, making the pump to automatically stop.

FIG. 2b shows a schematic side view of a drainage apparatus with a cuboid mechanism cover, together with a side and a top view of that mechanism cover. The cuboid mechanism cover is provided with one or more support(s) that support the drainage tubing.

FIG. 2c shows a schematic side view of a drainage apparatus with a slidable mechanism cover 231, together with a front and a top view of that mechanism cover. The housing 1 is provided with opening 233 for the slidable mechanism cover to slide. There is also arranged a lock release button 237 to release the slidable mechanism cover locking mechanism 235. The slidable mechanism cover 231 is provided with a semi-cylindrical inner surface to allow the loop of the drainage tubing to be compressed (pinched) between rollers of the mechanism and the semi-cylindrical inner surface.

The slidable mechanism cover 231 may preferably be provided with a switch that is configured to automatically turn off the pump when the slidable mechanism cover 231 is opened.

FIG. 2d shows a schematic side view of a drainage apparatus 200 with a hinged mechanism cover 241, together with a side and a top view of that mechanism cover. There is arranged a release button 245 to release cover lock mechanism. The hinged mechanism cover 241 is provided with a hinge 242 to allow for opening and closing of the cover 241. The hinged mechanism cover 241 is provided with a semi-cylindrical inner surface to allow the loop 3 of the drainage tubing to be compressed (pinched) between rollers of the mechanism and the semi-cylindrical inner surface.

The hinged mechanism cover 241 may preferably be provided with a switch that is configured to automatically turn off the pump when the hinged mechanism cover 241 is opened. The switch is preferably configured with a lever pivoted at the same hinge as the hinged mechanism cover 241, and with a contact arranged close to a lever end that when the hinged mechanism cover 241 is in a closed position, such that when the hinged mechanism cover 241 is correctly placed over the mechanism the contact is closed, and when the hinged mechanism cover 241 is moved away from its correct position the contact is opened, the switch is thus configured to break a pump circuit, making the pump to automatically stop when the cover is opened.

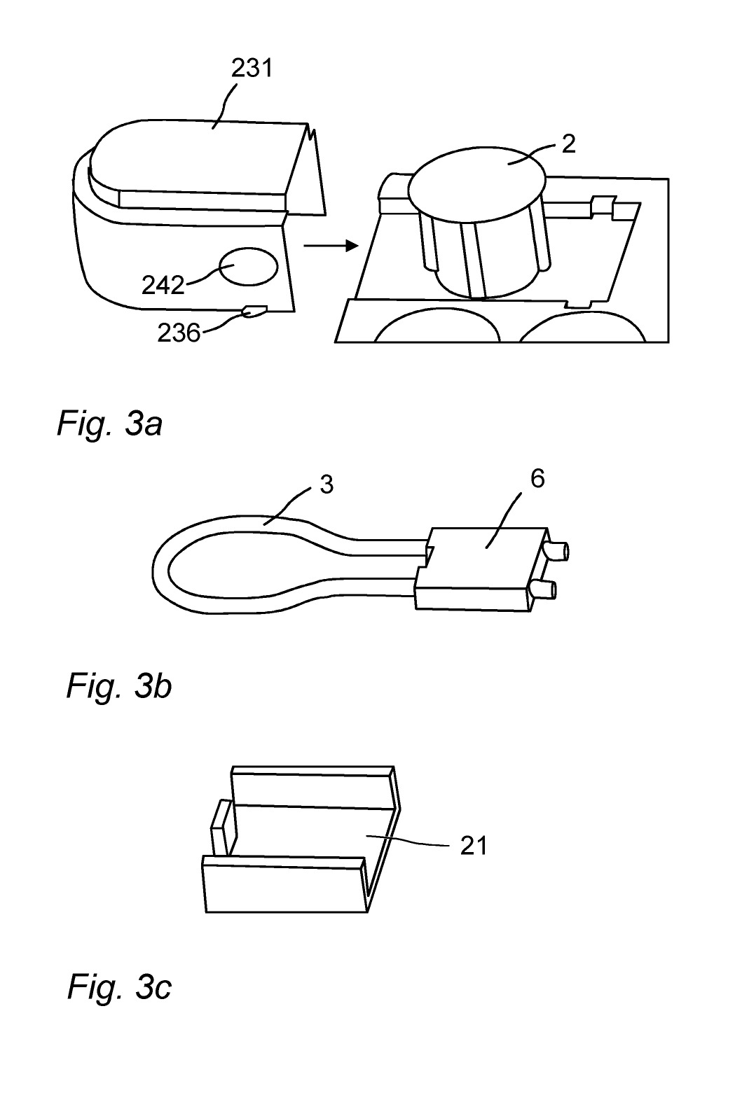

FIG. 3a shows a detail of the slideable mechanism cover 231 of FIG. 2c together with the peristaltic pump mechanism 2. A catch 236 of the locking mechanism 235 is arranged to firmly lock the cover into place when slided to the right in the figure. The locking mechanism is released by pushing locking mechanism release button 237. The cover is provided with a semi-cylindrical inner surface to allow the loop of the drainage tubing to be compressed (pinched) between rollers of the mechanism and the semi-cylindrical inner surface.

FIG. 3b shows a loop 3 of drainage tubing 3, 6, 7a, 7b and first fixation means 6. The tubing 3 is firmly attached to the fixation means 6 and do not allow for any sliding between them. The first fixation means 6 is preferably provided with mechanical protrusions and or indentations to enable the loop to be mounted in a certain position only, and making it impossible to mount the fixation means the wrong way in the second fixation means 21.

FIG. 3c shows second fixation means 21 being part of pump unit to receive first fixation means 6 of FIG. 3b. The second fixation means 21 is preferably provided with mechanical protrusions and/or indentations, inverted in relation to those of first fixation means 6, to enable first fixation means and the loop to be mounted in a certain position only, and making it impossible to mount the first fixation means 6 the wrong way in the second fixation means 21. This will secure and safeguard pumping in the wrong direction, which is an advantage if the drainage system is going to be operated by a patient who may lack deeper medical and/or technical skills, or may sometimes be a bit confused due to illness.

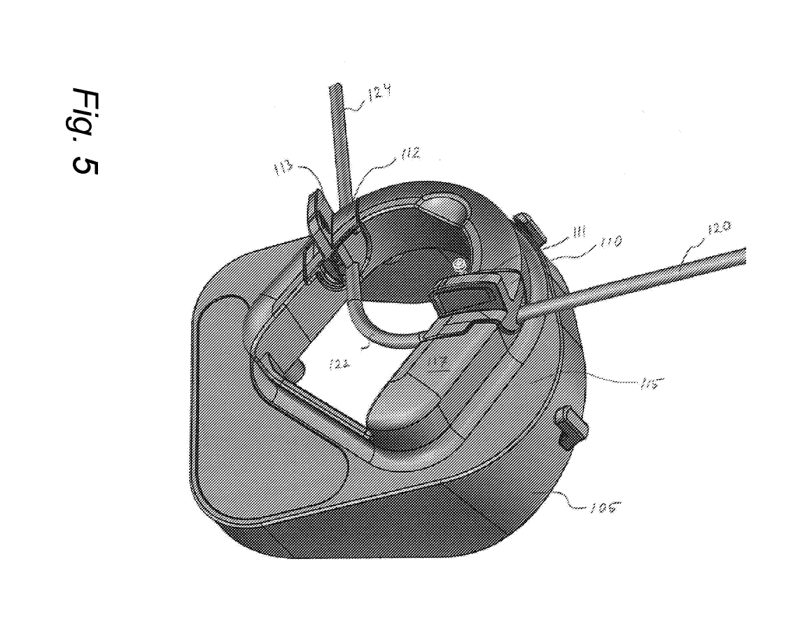

FIG. 4 shows the tube loop 3 of the drainage tubing 3, 6, 7 mounted around the peristaltic mechanism 2. It can also be seen how nicely the first fixation means 6 fits together with the second fixation means 21 to keep the drainage tube 3 in position for efficient pumping. When a cover having a cylindrical inner surface is locked into place, the peristaltic mechanism will be more efficient because the loop of the drainage tubing will be compressed (pinched) between rollers of the mechanism and the semi-cylindrical inner surface.

Length of Peristaltic Pump Portion of Tube

In the body drainage system, the pump portion 3 of the flexible tube unit 3, 6, 7 comprises a length of tube corresponding to a length of arc of 80 to 190 degrees of a rotational revolution of the peristaltic mechanism, such that the rollers of the peristaltic pump compresses this pump portion 3 during its course of action.

The pump portion 3 of the flexible tube unit 3, 6, 7 preferably comprises a length of tube corresponding to a length of arc of 80 to 140 degrees of a rotational revolution of the peristaltic mechanism, or more preferred, 80 to 100 degrees, or most preferred 85 to 95 degrees.

FIG. 5 shows fixation means of a drainage device for a 90-degree loop of drainage tubing. A frame 105, 115, 117 arranged around the pump mechanism are provided with fixation means 110, 112 for the drainage tubing at 90-degree angle to form a 90-degree loop of pump portion 122 of drainage tubing.

### Collection Bag Hanger

The body drainage system may further comprise a collection bag hanger (not shown) for hanging a collection bag for the drainage fluid, and wherein the collection bag hanger comprises weighing means to weigh the collection bag and its content in order to measure the collected volume, and wherein the weighing means comprises a load cell and/or a strain gauge. The weighing means may be arranged as part of a hanging portion of the hanger, i.e., measuring force extending the hanger. The weighing means may alternatively be arranged as part of a standing portion of the hanger, i.e., measuring compressive force.

### LEGEND

1. Housing
2 Peristaltic mechanism
3 Flexible tubing
4 Incoming flow connector
5 Outcoming flow connector
6 First fixation means of flexible tube
7 Drainage tube
7a Drainage tube first portion
7b Drainage tube second portion
8 Collection unit
9 Liquid property indicator
10 Peristaltic movement indicator
11 Peristaltic regulator
12 Power on button
13 Battery power indicator
14 Battery
15 Sample port of collection unit
21 Second fixation means of pump unit
105 Frame
115 Frame
117 Frame
110 Fixation means
112 Fixation means
122 Drainage tube pump portion
124 Drainage tube
200 Drainage apparatus
201 Cubic mechanism cover
202 Peristaltic mechanism
210 Cover stand
212 Cover release button
221 Cuboid mechanism cover
231 Slidable mechanism cover
233 Opening for cover to slide
235 Locking mechanism
236 Catch of locking mechanism
237 Locking mechanism release button
241 Hinged mechanism cover
242 Hinge
245 Hinged cover release button

1-27. (canceled)

28. A body drainage system for draining fluid from a body cavity of a patient, the body cavity being provided with an access port, wherein the system comprises
a rotatable peristaltic pump mechanism,
a housing for housing at least a portion of the peristaltic pump mechanism,
a flexible tube unit configured to be connected at an access port end to the patient access port, and at a collection unit end to a collection unit,
a cover for covering movable parts of the peristaltic pump mechanism
a regulator unit configured to regulate a rotational speed of the peristaltic pump mechanism, wherein the flexible tube unit is configured to form a loop suitable to be placed in one way only at peristaltic pump mechanism making it possible for the rotatable peristaltic mechanism to compress a pump portion of the flexible tube unit repeatedly such that pumping occurs in a desired direction.

29. A body drainage system according to claim 28, wherein the system further comprises
a cylindrical or partially cylindrical inner surface, wherein the loop of the flexible tube unit is configured to be placed in one way only at the rotatable peristaltic pump mechanism making it possible for the rotatable peristaltic mechanism to compress a pump portion of the flexible tube unit repeatedly against the cylindrical inner surface contributing to efficient pumping in the desired direction.

30. The drainage system according to claim 28, further comprising holding means that can be set in an open position, and that can be set in a closed position, respectively, and when set in the closed position provides the cylindrical inner surface mentioned in claim 28 for the peristaltic mechanism, and simultaneously constitutes the cover mentioned in claim 28.

31. The drainage system according to claim 28, wherein the regulator unit is configured to, upon user command, perform a drainage cycle, the drainage cycle comprising
an acceleration phase, during which the peristaltic pump accelerates from zero rpm to a first predetermined rpm, and
an operational phase, wherein the pump is controlled to keep the first rpm, and
a deceleration phase, during which the peristaltic pump decelerates from the first predetermined rpm to zero rpm.

32. The drainage system according to claim 28, wherein the flexible tube unit have a drainage tube first portion and a drainage tube second portion, and wherein the second portion 7b have an outlet end that can be connected to a collection unit, said unit being provided with chemical indication means, to provide the user with information regarding the chemical parameters of the fluid in the collection unit.

33. The drainage system according to claim 28, wherein the chemical indication means is color shifting indication means such as e.g. litmus paper.

34. A method for draining a bodily fluid via a drainage tube and a peristaltic pump, wherein the peristaltic mechanism is comprised of a rotating frame having a number of cylindrical compression rolls, and wherein the peristaltic mechanism and a cylindrical inner surface are configured to hold between them a loop of the flexible tube in order to compress the tube as the compression rolls travels along the loop, and wherein the cylindrical inner surface is arranged as an inner surface of a cover, arranged to hold the flexible tube in correct position against the peristaltic mechanism and to cover the peristaltic mechanism and prevent undesired objects like wet wipes and fingers to get caught in the mechanism.

35. The method of claim 34, wherein the bodily fluid is collected at an end of the flexible tube further analysis may be pH and lactate determined via chemical indicators.

36. The method of claim 34, wherein the flexible tube is directed to the correct position via fixation means that holds the flexible tube in position throughout the drainage procedure.

37. A method for drainage of bodily fluids, comprising a connection to an access port of a patient where bodily fluids is to be removed from a cavity, the method comprising the following steps:
inserting a flexible tube through a peristaltic pump mechanism so as to pump fluid into a collection unit, wherein a programmed loop of pumping is performed so as to perform a variety of applied suction pressure in the bodily cavity.

38. The method of claim 37, wherein the peristaltic movement is controlled by a pre-programmed regulating mechanism during operation.

39. The method of claim 37, wherein after insertion of the flexible tube and then closing a cover that holds the flexible tube into the peristaltic mechanism the peristaltic pump may be activated by pushing a power on button.

40. A kit of parts suitable for the drainage of a body fluid from a patient, the kit comprising:
a peristaltic pump unit, having a peristaltic mechanism, and first fixation means for a loop of drainage tubing;
a loop of drainage tubing having a second fixation means for cooperating with the first fixation means of the peristaltic pump unit in order to keep the drainage tubing in position during pumping;
a collection unit for collection the drained fluid.

41. A peristaltic pump unit, comprising:
a rotatable peristaltic pump mechanism,
a housing for housing at least a portion of the peristaltic pump mechanism,
a cover for covering movable parts of the peristaltic pump mechanism;
first fixation means for a loop of drainage tubing,
a regulator unit configured to regulate a rotational speed of the peristaltic pump mechanism.

42. A flexible tube unit configured to be connected at an access port end to a patient access port, and at a collection unit end to a collection unit, and further comprising second fixation means for cooperating with first fixation means of the peristaltic pump unit in order to keep the drainage tubing in position during pumping.

43. The flexible tube unit of claim 42 further comprising a collection bag provided with chemical indication means.
